(19) United States
(12) Patent Application Publication
(10) Pub. No.: US 2024/0225650 A1
(43) Pub. Date: Jul. 11, 2024

(54) APPARATUS FOR ENDOLUMINAL ACCESS TO GASTRO-INTESTINAL TRACT

(71) Applicant: BariaTek Medical, Paris (FR)

(72) Inventor: Youssef BIADILLAH, Maisons-Laffitte (FR)

(73) Assignee: BariaTek Medical, Paris (FR)

(21) Appl. No.: 18/563,050

(22) PCT Filed: Jun. 21, 2022
(86) PCT No.: PCT/EP2022/066862

§ 371 Date: Nov. 21, 2023

(30) Foreign Application Priority Data
Jun. 22, 2021 (EP) 21315099.8

(51) Int. Cl. A61B 17/11; A61B 90/00

(52) U.S. Cl. CPC A61B 17/1114; A61B 90/06; A61B 90/39; A61B 2017/1121; A61B 2090/061; A61B 2090/3912; A61B 2090/3966

(57) ABSTRACT

Gastro-intestinal-tract endoluminal apparatus (10) for deploying markers (42, 44, 46) into a gastro-intestinal tract of a patient, the apparatus being retrievably insertable into the gastro-intestinal tract, comprising: a flexible tube (12) introducible into the gastro-intestinal tract of a patient, the tube having a stationary end (12d) and a mobile lumen (14), the tube being extendable within the tract by inflating an inflatable region (16) of the tube around the lumen, the inflation causing distal-most invaginated material (12c) to evert outwardly such that the everted material extends a deployed length of the tube distally along a path of the gastro-intestinal tract, invaginated material (12b) of the lumen being drawn distally within the deployed tube to feed the distal extension.

### FIELD OF THE INVENTION

The present invention relates to the field of endoluminal access to a duct or lumen within a patient's body, especially but not exclusively, the gastro-intestinal tract. Some embodiments relate to access for assisting creation of an anastomosis in the gastro-intestinal tract or other body duct.

### BACKGROUND TO THE INVENTION

An anastomosis is a surgical cross-connection or bridge between two different sections of body duct lumen. The gastro-intestinal tract is the luminal route in the body from the oesophagus to the anus. Anastomoses formed somewhere along or in the gastrointestinal tract are one form of therapy used to treat digestion-related problems, such as diabetes, obesity, bowel diseases and obstructions. An anastomosis can be used to bypass a portion of the gastro-intestinal tract, such as a portion of the small intestine, to avoid sensitive areas or to influence or reduce absorption of nutrients.

Currently, open-surgery provides most comprehensive access to the internal anatomy for forming an anastomosis. However, open-surgery is highly invasive, and unsuitable for many patients and conditions to be treated. Minimally invasive procedures have been proposed, but significant challenges remain in forming anastomoses equally effectively by a minimally invasive procedure, especially endoluminally. In an endoluminal procedure, one or more tools are introduced into the body principally through the body duct in which the anastomosis is to be made. Current endoluminal techniques are best suited to anastomosis procedures that are relatively shallow in the body duct. This limits, for example, the versatility of the procedure for the small intestine which, in most adults, can have a length of up to 6 or 7 meters, and is folded to follow a highly tortuous path in the abdomen.

### SUMMARY OF THE INVENTION

It would be desirable to address and/or mitigate one or more of the above issues.

Aspects of the invention are defined in the claims.

Additionally or alternatively, one aspect of the invention provides gastro-intestinal-tract endoluminal apparatus for assisting creation of an anastomosis between spaced apart positions in the gastro-intestinal tract of a patient. The apparatus is or can be retrievably insertable into the gastro-intestinal tract, and is able to follow a curved path of the small intestine.

The apparatus comprises a flexible tube introducible into the gastro-intestinal tract of a patient, with the tube in an at least partly invaginated state, the tube having a stationary end and a mobile lumen. The tube is extendable within the tract without substantial movement of the stationary end, by inflating an inflatable region of the tube around the lumen. The inflation causes distal-most invaginated material to evert outwardly such that the everted material extends a deployed length of the tube distally along a path of the gastro-intestinal tract. Invaginated material of the lumen is drawn distally within the deployed tube to feed the distal extension.

Such an arrangement can provide significant advantages compared to a conventional catheter or endoscopic apparatus.

One advantage is the reduction in frictional sliding contact between the apparatus and the gastro-intestinal tract tissue. The tube has a stationary end that is generally immobile. Extension of the deployed length of tube is achieved by everting invaginated material from within the tube, rather than by advancing the exterior of the tube. As a result, there is very little or no frictional sliding resistance as the tube advances, even deep with the tract, and even in the tortuous turns of, for example, the small intestine. In contrast, a conventional catheter or endoscope comprises a tubular shaft with an exterior surface that slides against the tissue wall as it advances. The frictional resistance increases the further the shaft is advanced within the tract and contacts progressively more and more tissue. The frictional resistance also increases with tortuosity of the tract, which can limit the depth to which some conventional devices can be navigated within, for example, the small intestine.

Another advantage is that the tube is highly conformable and can advance with ease through folded and tortuous turns of, for example, the small intestine. In contrast, a conventional catheter or endoscope has a shaft that has more resistance to bending, and as a result can limit the depth to which some conventional devices can be navigated.

In some embodiments, the tube is retrievable by drawing an inner portion of the tube, optionally the lumen, in a proximal direction to collapse and/or invaginate the tube from its distal-most region. Such a withdrawal technique can achieve the same advantages during withdrawal as those discussed above for introduction.

When preparing the target site or sites for an anastomosis, the present invention can greatly assist in measuring distances between certain points in the gastro-intestinal tract, to ensure that a certain distance in the tract will be bypassed. Additionally or alternatively, positions can be marked to facilitate anastomosis preparation and creation.

In some embodiments, the tube comprises at least one marker element for marking a predetermined length or position along the deployed portion of the tube. Optionally, the marker element is radio-opaque to facilitate detection by fluoroscopic imaging. Additionally or alternatively, the marker element may be expandable from a collapsed configuration to an expanded configuration.

Additionally or alternatively to a marker element on the tube, the apparatus may further comprise a guidewire insertable though the lumen towards a distal end of the tube. The guidewire may be configured to remain in place when the tube is retrieved.

In some embodiments, the guidewire carries at least one marker element for marking a predetermined length or position along the guidewire. Optionally, the marker element is radio-opaque to facilitate detection by fluoroscopic imaging.

In some embodiments, the marker element is expandable from a collapsed condition on the guidewire, to an expanded condition laterally larger than a main portion of the guidewire. The marker element may comprise (i) an anchor for expanding against tissue of the gastro-intestinal tract, and/or (ii) an expandable cage.

Howsoever the at least one marker element is implemented on the tube and/or on a guidewire, a plurality of said marker elements may be provided. First and second marker elements may spaced apart by a predetermined distance. Additionally or alternatively, at least some of the marker elements are spaced apart by a uniform repeating separation.

Additionally or alternatively to any of the above, in some embodiments, the lumen comprises a non-evertable region that is pulled distally within the tube as the tube extends, optionally until the non-evertable region reaches the distal-most portion of the tube. The non-evertable region may define a working channel for insertion of a guidewire and/or one or more tools.

A second aspect of the invention, optionally in combination with any of the features of the first aspect, may provide gastrointestinal-tract endoluminal apparatus for deploying markers into a gastro-intestinal tract of a patient, the apparatus being retrievably insertable into the gastro-intestinal tract, and able to follow a curved path of the small intestine. The apparatus may comprise:
a flexible tube introducible into the gastro-intestinal tract of a patient with the tube in an at least partly invaginated state, the tube having a stationary end and a mobile lumen, the tube being extendable within the tract without substantial movement of the stationary end, by inflating an inflatable region of the tube around the lumen, the inflation causing distal-most invaginated material to evert outwardly such that the everted material extends a deployed length of the tube distally along a path of the gastro-intestinal tract, invaginated material of the lumen being drawn distally within the deployed tube to feed the distal extension; and
a plurality of marker elements detectable by fluoroscopic imaging, optionally wherein at least one of the marker elements is deployable from a collapsed state to an expanded state.

At least one of the marker elements may be carried by the tube. Additionally or alternatively, the apparatus may further comprise a guidewire insertable via the lumen of the tube, the guidewire carrying at least one of the marker elements.

A third aspect of the invention provides a method of introducing an apparatus into the gastro-intestinal-tract of a patient. The apparatus may optionally include any of the features described in the first and second aspects. Additionally or alternatively, the apparatus can comprise a flexible tube introducible into the gastro-intestinal tract of a patient with the tube in an at least partly invaginated state, the tube having a stationary end and a mobile lumen,

The method comprises inflating an inflatable region of the tube around the lumen, the inflation causing distal-most invaginated material to evert outwardly such that the everted material extends a deployed length of the tube distally along a path of the gastro-intestinal tract, invaginated material of the lumen being drawn distally within the deployed tube to feed the distal extension, whereby the tube is extendable within the tract without substantial movement of the stationary end.

In some embodiments, the method comprises, or further comprises, deploying at least one marker element for indicating a predetermined length or position. The deployment step may optionally occur as part of and/or as a consequence of, inflation of the tube as the tube extends progressively. Additionally or alternatively, the deployment step may be or comprise a step additional to inflation of the tube.

The marker element may optionally be radio-opaque to facilitate identification by medical imaging techniques. The step of deploying a marker element may comprise causing the marker element to expand.

In some embodiments, the method comprises or further comprises deploying at least a second marker element.

Another aspect of the invention provides a method of identifying a target position in a gastro-intestinal-tract of a patient, the method comprising:
providing apparatus comprising a flexible tube introducible into the gastro-intestinal tract of a patient with the tube in an at least partly invaginated state, the tube having a stationary end and a mobile lumen, the apparatus optionally according to the first and/or second aspect above,
inflating an inflatable region of the tube around the lumen, the inflation causing distal-most invaginated material to evert outwardly such that the everted material extends a deployed length of the tube distally along a path of the gastro-intestinal tract, invaginated material of the lumen being drawn distally within the deployed tube to feed the distal extension, and
causing deployment of at least one marker element for identifying the target site.

Another aspect of the invention provides a method of measuring a distance in a gastro-intestinal-tract of a patient, the method comprising:
providing apparatus comprising a flexible tube introducible into the gastro-intestinal tract of a patient with the tube in an at least partly invaginated state, the tube having a stationary end and a mobile lumen, the apparatus optionally according to the first and/or second aspect above,
inflating an inflatable region of the tube around the lumen, the inflation causing distal-most invaginated material to evert outwardly such that the everted material extends a deployed length of the tube distally along a path of the gastro-intestinal tract, invaginated material of the lumen being drawn distally within the deployed tube to feed the distal extension, and
causing deployment of at least a first marker element and a second marker element having a predetermined separation.

In any of the above aspects, the apparatus may be, or may be configured to be, introduced into an intestine of a patient through the patient's mouth and/or stomach.

Another aspect of the invention extends use of the invention to use with other ducts within a patient's body. Apparatus may be provided for assisting creation of an anastomosis between spaced apart positions in body duct a patient, the apparatus being retrievably insertable into the duct (e.g. endoluminally), and able to follow a curved path of the duct. The apparatus may comprise: a flexible tube introducible into the duct of a patient with the tube in an at least partly invaginated state, the tube having a stationary end and a mobile lumen, the tube being extendable within the body duct without substantial movement of the stationary end, by inflating an inflatable region of the tube around the lumen, the inflation causing distal-most invaginated material to evert outwardly such that the everted material extends a deployed length of the tube distally along a path of the body duct, invaginated material of the lumen being drawn distally within the deployed tube to feed the distal extension.

Optionally, this aspect may use any of the features described for the preceding aspects.

It will be appreciated that, in any of the above aspects, the distal direction is a direction extending deeper along the body duct or tract with respect to a point of entry of the apparatus into the body duct or tract, and/or with respect to the stationary end of the tube, and/or with respect to an operator of the apparatus.

Although certain ideas, features and advantages have been highlighted above and in the appended claims, protection is claimed for any novel feature or idea described herein and/or illustrated in the drawings, whether or not emphasis has been placed thereon.

### BRIEF DESCRIPTION OF THE DRAWINGS

FIG. 1 is a schematic section illustrating an invaginated tube apparatus.

FIG. 2 is a schematic section illustrating extension of an invaginated tube apparatus to increase its deployed length.

FIG. 3 is a schematic section illustrating full deployment of the tube.

FIG. 4 is a schematic section illustrating introduction of the tube apparatus into the gastro-intestinal tract.

FIG. 5 is a schematic section illustrating deployment of the tube into the small intestine.

FIG. 6 is a schematic section illustrating a guidewire placed in the small intestine by introduction through the deployed tube.

FIG. 7 is a schematic section illustrating a further example of guidewire with expandable anchors.

### DETAILED DESCRIPTION OF PREFERRED EMBODIMENTS

Non-limiting embodiments are now described by way of example, with reference to the accompanying drawings. The same reference numerals denote the same or equivalent features whether or not described explicitly in detail.

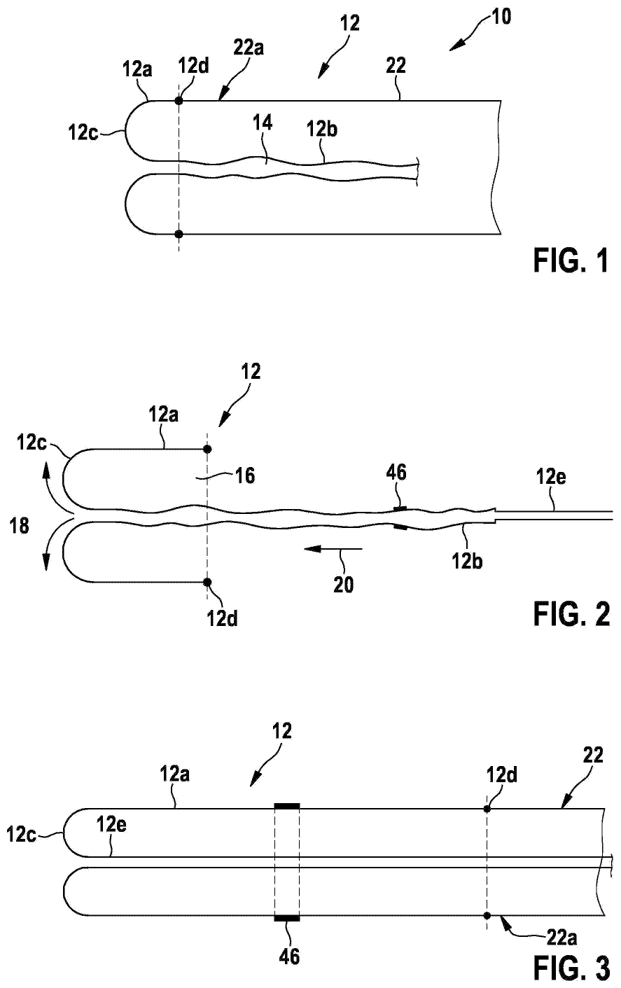

FIGS. 1 to 3 illustrate the deployment principles of one embodiment of a gastro-intestinal-tract apparatus 10 configured for assisting creation of an anastomosis between spaced apart positions in the gastro-intestinal tract of a patient, and able to follow a curved path of the small intestine.

The apparatus comprises a flexible tube 12 introducible into the gastro-intestinal tract of a patient with the tube in an at least partly invaginated state. The tube 12 includes everted external material 12a, invaginated material 12b defining a lumen 14, distal-most invaginated material 12c at the distal tip of the tube and communicating with the everted material 12a and the invaginated material 12b, and a generally stationary end 12d.

In use, the tube 12 is extendable within the gastro-intestinal tract by applying inflation pressure within an inflatable region 16 of the tube 12 around the lumen 12b. Any suitable inflation fluid may be used, for example, a liquid (such as saline) or a gas (such as air). Referring to FIG. 2, inflation causes distal-most invaginated material 12c to evert outwardly (represented by arrows 18) such that the everted material extends a deployed length of the tube distally. Invaginated material 12b of the lumen 14 is drawn distally to feed the distal extension (represented by arrow 20). The invaginated material 12b includes sufficient excess material that the tube can adopt the enlarged, everted diameter without substantial elastic stretching. The excess material may be loosely folded at the lumen 14.

Optionally, a section of the lumen may further comprise a non-evertable region 12e, for example, a region having a transverse dimension insufficient to evert outwardly. When the non-evertable region reaches the distal-most portion of the tube, further distal extension is stopped. In other words, the tube 12 has reached a fully deployed condition (FIG. 3).

An advantage of the deployment technique for the tube 12 is that that the tube can extend within the gastro-intestinal tract with very little or substantially no frictional resistance. The exterior of the tube 12 does not substantially slide against surrounding body tissues, instead it remains stationary with respect to the stationary end 12d. Also, the absence of a central support or spine results in the tube 12 being very flexible and conformable, able to extend along and around bends in the unpredictable and tortuous path of, for example, the small intestine.

The apparatus may optionally further comprise a sheath 22 carrying the tube 12. The stationary end 12d of the tube 12 may, for example, be attached near or at a distal region 22a of the sheath 22, the adjacent invaginated tube material 12b being accommodated within the sheath 22 and extending proximally with respect to the distal region 22a of the sheath 22. The sheath 22 may facilitate initial introduction of the tube 12 into the gastro-intestinal tract, and may define part of the conduit passage in combination with and/or collectively with the tube.

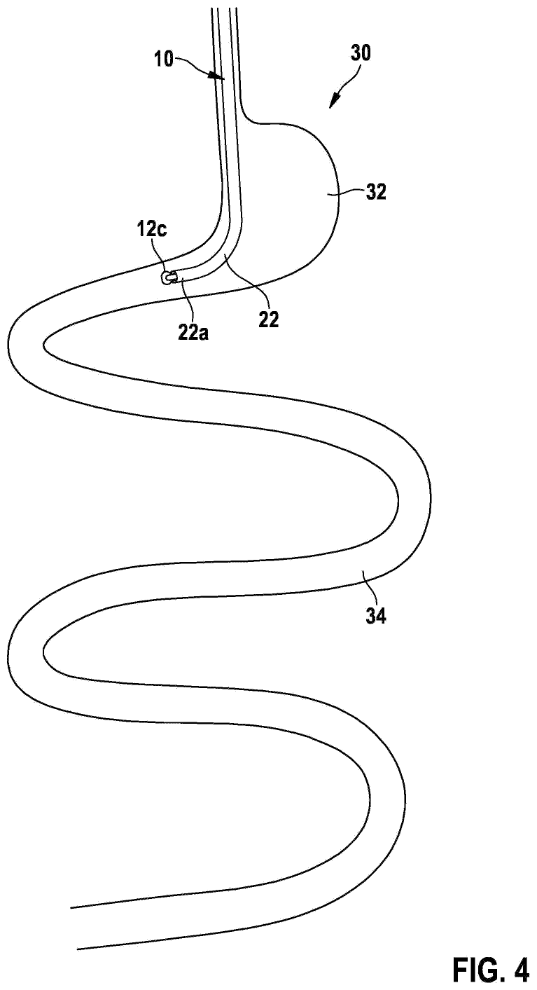

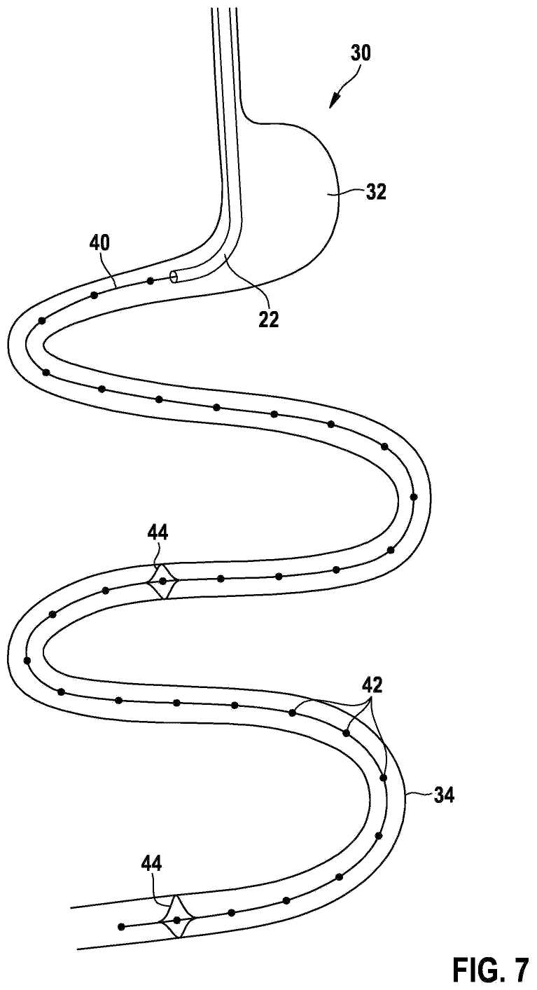

FIGS. 4-7 illustrate working examples of the apparatus 10.

Referring to FIG. 4, the apparatus 10 is introduced into the gastro-intestinal tract 30 of a patient, for example, via the mouth and oesophagus, through the stomach 32 towards the small intestine 34. Navigating the apparatus 10 through the oesophagus and stomach 32 is relatively straightforward, because the gastro-intestinal tract is relatively large and not tortuous. This part of the introduction may be performed by the sheath 22, which may have a length to extend to about the bottom of the stomach 32. In this initial stage, the invaginated tube 12 is received at least partly within, and extends proximally within, the sheath 22. The tube 12 does not project substantially beyond the distal end region 22a of the sheath 22, only a small distal-most invaginated region 12c of the tube being depicted in FIG. 4.

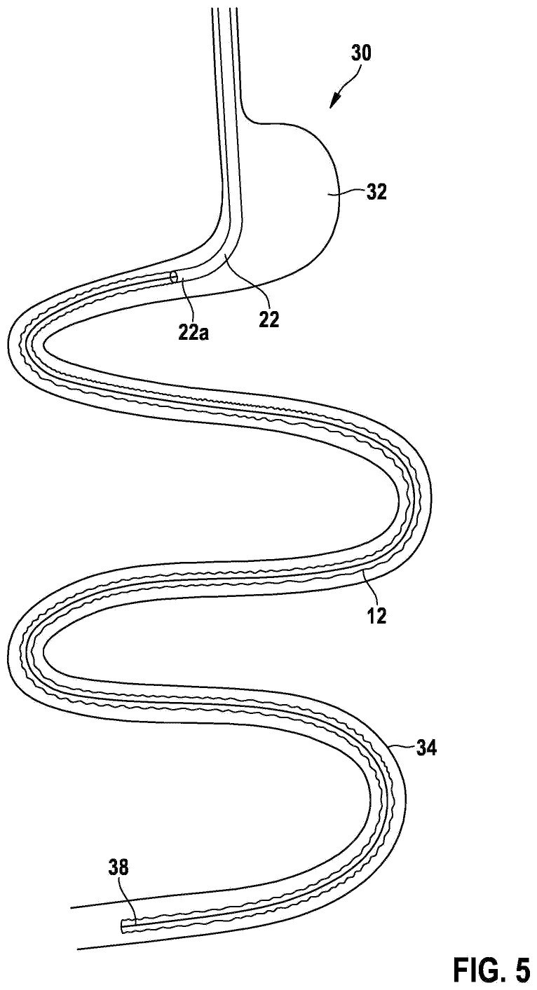

Referring to FIG. 5, the tube 12 is extended into the small intestine 34 by inflating the tube 12 to evert distal-most invaginated material, as described above. The deployed length of the tube 12 extends as more invaginated material everts from the tip, until the tube 12 reaches its fully deployed state illustrated in FIG. 5. A lumen formed by a non-evertable portion of material defines a working channel 38 extending from the distal end of the tube 12 to the distal end 22a of the sheath 22, the working channel further extending within the sheath 22 to the sheath proximal end (not shown), optionally outside the body.

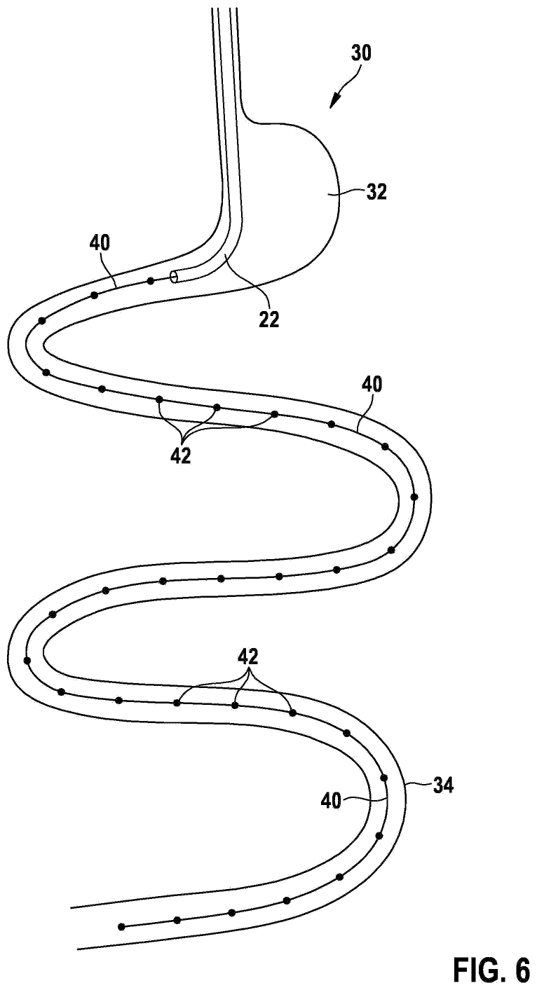

Referring to FIG. 6, the apparatus 10 further comprises a guidewire 40 insertable through the working channel 38. Thereafter, the tube 12 can be withdrawn by pulling on the tube proximal end, in order to collapse the tube inwardly from its distal end. The guidewire 40 may remain in place in the small intestine, as shown in FIG. 6. Optionally, the sheath 22 may remain within the oesophagus and stomach 32.

When preparing a target site or sites for an anastomosis, the guidewire 40 can greatly assist in measuring distances between certain points in the gastro-intestinal tract 30, to ensure that a certain distance in the tract will be bypassed. For example, the guidewire may carry at least one, optionally at least two, optionally three or more marker elements 42 for marking a predetermined length or position along the guidewire 40. The marker elements are preferably radio-opaque to facilitate detection by fluoroscopic imaging. The marker elements 42 may comprises different material (e.g. more radio-opaque) than the guidewire 40, and/or the marker elements 42 may be larger so as to be identifiable. In the illustrated example, the marker elements 42 are generally uniformly distributed along the length of the guidewire 40, with a uniform separation between adjacent marker elements 42, to enable distance to be measured by counting the marker elements 42.

Additionally or alternatively to the marker elements 42, one or more deployable marker elements 44 may be provided on the guidewire 40. For example, the deployable marker elements 44 may be in the form of deployable anchors or cages. The deployable marker elements 44 may, for example, be self-expandable when the tube 12 is removed from the guidewire 40, the deployable marker elements 44 may be manually deployed by the operator using a remote deployment mechanism (not shown). First and second deployable marker elements 44 are illustrated, set a predetermined distance apart for identifying positions to be joined together by an anastomosis achieving a predetermined bypass length. The deployable marker elements 44 may made of radio-opaque material to facilitate detection by fluoroscopy.

Whether or not a guidewire 40 is used, optionally one or more marker elements 46 may also be disposed on the tube 12. The or each marker element 46 is collapsible and expandable with the material of the tube, and expands outwardly when the portion of the tube 12 carrying the marker element 46 everts outwardly to add to the deployed length of the tube 12. The or each marker element 46 may comprise radio-opaque material to facilitate detection by fluoroscopy. Plural marker elements 46 may define measurement demarcations in a similar manner to the marker elements 42 described above, or predetermined position markers similar to the marker elements 44 described above.

Although the above embodiments have been described in the context of a gastro-intestinal-tract endoluminal apparatus, and the embodiments provide significant advantages in facilitating access deep within the small intestine, and for deploying marker elements to assist with anastomosis creation, it will be appreciated that the concepts can be applied more broadly to other body ducts, in particular following a curved or tortuous path.

It will be appreciated that the foregoing description is merely illustrative of example embodiments of the invention, and that many modifications and equivalents may be used within the principles of the invention.

1-28. (canceled)

29. A gastro-intestinal-tract endoluminal apparatus for assisting creation of an anastomosis between spaced apart positions in the gastro-intestinal tract of a patient, the apparatus being retrievably insertable into the gastro-intestinal tract, and able to follow a curved path of the small intestine, comprising:
a flexible tube introducible into the gastro-intestinal tract of a patient with the tube in an at least partly invaginated state, the tube having a stationary end and a mobile lumen, the tube being extendable within the tract without substantial movement of the stationary end, by inflating an inflatable region of the tube around the lumen, the inflation causing distal-most invaginated material to evert outwardly such that the everted material extends a deployed length of the tube distally along a path of the gastro-intestinal tract, invaginated material of the lumen being drawn distally within the deployed tube to feed the distal extension.

30. The apparatus according to claim 29, wherein the tube is retrievable by drawing an inner portion of the tube in a proximal direction to collapse and/or invaginate the tube from its distal-most region.

31. The apparatus according to claim 29, further comprising a sheath coupled to the stationary end of the tube, for facilitating introduction of the apparatus into the gastro-intestinal tract.

32. The apparatus according to claim 29, wherein the tube comprises at least one marker element for marking one of a predetermined length or position along the deployed portion of the tube.

33. The apparatus according to claim 32, wherein the marker element is radio-opaque to facilitate detection by fluoroscopic imaging.

34. The apparatus according to claim 32, wherein the marker element is expandable from a collapsed configuration to an expanded configuration.

35. The apparatus according to claim 29, wherein the lumen comprises a non-evertable region (12e) that is pulled distally within the tube as the tube extends, the non-evertable region defining a working channel for insertion of a guidewire and/or one or more tools.

36. The apparatus according to claim 29, further comprising a guidewire insertable though the lumen towards a distal end of the tube.

37. The apparatus according to claim 36, wherein the guidewire carries at least one marker element for marking a predetermined length or position along the guidewire.

38. The apparatus according to claim 37, wherein the marker element is radio-opaque to facilitate detection by fluoroscopic imaging.

39. The apparatus according to claim 37, wherein the marker element is expandable from a collapsed condition on the guidewire, to an expanded condition laterally larger than a main portion of the guidewire.

40. The apparatus according to claim 39, wherein the marker element comprises (i) an anchor for expanding against tissue of the gastro-intestinal tract, and/or (ii) an expandable cage.

41. The apparatus according to claim 32, comprising a plurality of said marker elements.

42. The apparatus according to claim 41, wherein first and second marker elements are spaced apart by a predetermined distance.

43. Apparatus according to claim 41, wherein at least some of the markers are spaced apart by a uniform repeating separation.

44. The apparatus according to claim 29, configured to be introduced into an intestine of a patient through the patient's stomach.

45. A method of introducing an apparatus into the gastro-intestinal-tract of a patient, the apparatus comprising a flexible tube introducible into the gastro-intestinal tract of a patient with the tube in an at least partly invaginated state, the tube having a stationary end and a mobile lumen, the method comprising:
inflating an inflatable region of the tube around the lumen, the inflation causing distal-most invaginated material to evert outwardly such that the everted material extends a deployed length of the tube distally along a path of the gastro-intestinal tract, invaginated material of the lumen being drawn distally within the deployed tube to feed the distal extension, whereby the tube is extendable within the tract without substantial movement of the stationary end.

46. The method according to claim 45, further comprising a step of deploying at least one marker element for indicating a predetermined length or position.

47. The method according to claim 46, wherein the step of deploying a marker element comprises causing the marker element to expand.

48. The method according to claim 46, further comprising a step of deploying at least a second marker element.

49. The method according to claim 45, further comprising a step of introducing the tube into the gastro-intestinal tract through the patient's mouth and stomach.

50. A method of identifying a target position in a gastro-intestinal-tract of a patient, the method comprising:
providing apparatus comprising a flexible tube introducible into the gastro-intestinal tract of a patient with the tube in an at least partly invaginated state, the tube having a stationary end and a mobile lumen,
inflating an inflatable region of the tube around the lumen, the inflation causing distal-most invaginated material to evert outwardly such that the everted material extends a deployed length of the tube distally along a path of the gastro-intestinal tract, invaginated material of the lumen being drawn distally within the deployed tube to feed the distal extension, and
causing deployment of at least one marker element for identifying the target site.

51. A method of measuring a distance in a gastro-intestinal-tract of a patient, the method comprising:
providing apparatus comprising a flexible tube introducible into the gastro-intestinal tract of a patient with the tube in an at least partly invaginated state, the tube having a stationary end and a mobile lumen,
inflating an inflatable region of the tube around the lumen, the inflation causing distal-most invaginated material to evert outwardly such that the everted material extends a deployed length of the tube distally along a path of the gastro-intestinal tract, invaginated material of the lumen being drawn distally within the deployed tube to feed the distal extension, and
causing deployment of at least a first marker element and a second marker element having a predetermined separation.
